(19) United States
(12) Patent Application Publication
(10) Pub. No.: US 2025/0194902 A1
(43) Pub. Date: Jun. 19, 2025

(54) RELAY ADAPTER AND MEDICAL APPARATUS SYSTEM

(71) Applicant: OLYMPUS MEDICAL SYSTEMS CORP., Tokyo (JP)

(72) Inventor: Takeo SUZUKI, Tokyo (JP)

(73) Assignee: OLYMPUS MEDICAL SYSTEMS CORP., Tokyo (JP)

(21) Appl. No.: 18/977,086

(22) Filed: Dec. 11, 2024

Related U.S. Application Data
(60) Provisional application No. 63/609,894, filed on Dec. 14, 2023.

(51) Int. Cl. A61B 1/00; A61B 1/06; A61B 1/07

(52) U.S. Cl. CPC A61B 1/00128; A61B 1/00029; A61B 1/00103; A61B 1/00124; A61B 1/00126; A61B 1/00016; A61B 1/0669; A61B 1/07

(57) ABSTRACT

A relay adapter includes a first adapter including a first end side configured to connect to a first receptacle of a power supply apparatus, a second end side, a power supply circuit configured to supply power transmitted from the power supply apparatus to the second end side, and a detection circuit, and a second adapter including a first end side configured to connect to the second end side of the first adapter, a second end side configured to connect to a connector of a medical apparatus, and an electric circuit configured to transmit power supplied to the first end side of the second adapter from the first adapter to the connector of the medical apparatus, the detection circuit is configured to detect whether the second end side of the first adapter is connected to the first end side of the second adapter.

### CROSS-REFERENCE TO RELATED APPLICATION

This application is based on and claims priority under 35 U.S.C. § 119 to U.S. Provisional Application No. 63/609,894, filed on Dec. 14, 2023, the entire contents of which are incorporated herein by reference.

### BACKGROUND

### 1. Field

The present disclosure relates to a relay adapter configured to connect a power supply apparatus and a medical apparatus, and a medical apparatus system including the relay adapter.

### 2. Description of the Related Art

Conventionally, reusable endoscopes, which are configured to be used a plurality of times after being cleaned and disinfected, have been used as medical apparatuses. Such endoscopes are each configured such that a connector of the endoscope is connected to a receptacle of an endoscope processor or the like. The endoscope processor functions as a power supply apparatus and is configured to supply power to the endoscope. In addition, the endoscope processor performs image processing and the like on an image pickup signal received from the endoscope.

When a reusable endoscope is cleaned and disinfected, a waterproof cap for protecting an electric contact has to be attached to a connector of the endoscope, for example.

In view of the above, Japanese Patent Application Laid-Open Publication No. 2016-067535 discloses an endoscope with a connector, which can perform electric power reception, control signal communication, and image signal communication in non-contact manner. The publication further discloses a configuration in which the connector can be separated into a plurality of connector cases for enabling easy assembly, repair, and maintenance of the connector.

In contrast to a reusable endoscope that can be used a plurality of times, a single-use endoscope, which is disposed of after a single use, has been proposed. Such a single-use endoscope sometimes employs a standard different from that of a conventional reusable endoscope. For example, the single-use endoscope sometimes uses components of a universal standard for cost reduction.

For such a single-use endoscope of a standard different from that of a conventional reusable endoscope, an endoscope processor, a light source system, and the like, which are designed exclusively for such a single-use endoscope, are used, for example.

### SUMMARY

A relay adapter according to one aspect of the present disclosure includes: a first adapter including: a first end side configured to connect to a first receptacle of a power supply apparatus; a second end side; a power supply circuit configured to supply power transmitted from the power supply apparatus to the second end side; and a detection circuit; and a second adapter including: a first end side configured to connect to the second end side of the first adapter; a second end side configured to connect to a connector of a medical apparatus; and an electric circuit configured to transmit power supplied to the first end side of the second adapter from the first adapter to the connector of the medical apparatus, wherein the detection circuit is configured to detect whether the second end side of the first adapter is connected to the first end side of the second adapter.

A medical apparatus system according to one aspect of the present disclosure includes: a medical apparatus including a connector; a power supply apparatus including a first receptacle, and the power supply apparatus configured to transmit power to be supplied to the medical apparatus; and a relay adapter including: a first adapter including: a first end side configured to connect to a first receptacle of a power supply apparatus; a second end side; a power supply circuit configured to supply power transmitted from the power supply apparatus to the second end side; and a detection circuit; and a second adapter including; a first end side configured to connect to the second end side of the first adapter; a second end side configured to connect to a connector of a medical apparatus; and an electric circuit configured to transmit power supplied to the first end side of the second adapter from the first adapter to the connector of the medical apparatus; the detection circuit is configured to detect whether the second end side of the first adapter is connected to the first end side of the second adapter.

### BRIEF DESCRIPTION OF THE DRAWINGS

FIG. 1 is a view showing a configuration of an endoscope system of a first embodiment according to the present disclosure.

FIG. 2 is a partially exploded perspective view showing a configuration for connecting an endoscope and an endoscope processor by using a relay adapter in the first embodiment.

FIG. 3 is a perspective view showing a first coupler side of a first adapter in the first embodiment.

FIG. 4 is a perspective view showing a second coupler side of a second adapter in the first embodiment.

FIG. 5 is a perspective view showing a first connector of the endoscope in the first embodiment.

FIG. 6 is a perspective view showing a first example in which only the first adapter of the relay adapter is connected to a first receptacle in the first embodiment.

FIG. 7 is a perspective view showing a second example in which only the first adapter of the relay adapter is connected to the first receptacle in the first embodiment.

FIG. 8 is a block diagram showing an electrical configuration of the relay adapter in the first embodiment.

### DETAILED DESCRIPTION

In general, there is a demand for reducing a cost for purchasing a new apparatus by using an endoscope processor, a light source system and the like, which have been used for a conventional reusable endoscope, for a single-use endoscope.

For example, in a low-cost single-use endoscope, in particular, not only standards of signals and the like but also a standard of hardware of a connector for connection to an endoscope processor are different from those in a reusable endoscope in some cases. The connector of the low-cost single-use endoscope has a simple configuration compared with the connector of the reusable endoscope.

When a single-use endoscope of a standard different from that of a reusable endoscope is connected to an endoscope processor for the reusable endoscope, the connection is required to be made via a relay adapter. Here, the single-use endoscope is supposed to be used once, while the relay adapter is supposed to be used a plurality of times.

In applications for a plurality of cases, single-use endoscopes are supposed to be used for a greater number of cases per day than reusable endoscopes. In such applications, it is supposed that only the single-use endoscopes are attached and detached, while the relay adapter is left inserted in the receptacle of the endoscope processor.

In general, in a case where an endoscopy room is narrow, for example, a user may have an impression that the relay adapter is protruded largely from the endoscope processor. Furthermore, if the relay adapter is protruded largely, a space required for a procedure in the endoscopy room may possibly limited.

Meanwhile, in order to prevent the protrusion of the relay adapter from the endoscope processor, it can be considered that the relay adapter 5 is detached from the endoscope processor each time after the single-use endoscope is used. However, in this case, a burden of a work for a user to perform will increase. In addition, if the number of cases for which single-use endoscopes are used is larger than the number of cases for which the conventional reusable endoscope is used, the number of attaching and detaching the relay adapter to and from the receptacle of the endoscope processor becomes larger than before. Then, it can be considered that the receptacle of the endoscope processor degrades faster than the conventional one, which causes a concern about an increase in repair frequency of the endoscope processor.

According to an embodiment to be described below, it is possible to provide a relay adapter which is prevented from protruding from a receptacle of an endoscope processor, and a medical apparatus system including the relay adapter.

Hereinafter, embodiments of the present disclosure will be described with reference to drawings. However, the present disclosure is not limited by the embodiment to be described below.

Note that in the description in the drawings, the same or corresponding elements are attached with the same reference signs as appropriate. In addition, the drawings are schematic, and care should be taken to the fact that a relationship among the lengths of the respective elements, a ratio of the length of a certain element to that of another element, the number of the respective elements, and the like are different from the actual ones for simplification of the description. Furthermore, the respective drawings sometimes include parts in which the relationships and ratios among the lengths of the elements, the number of the elements, and the like are different.

### First Embodiment

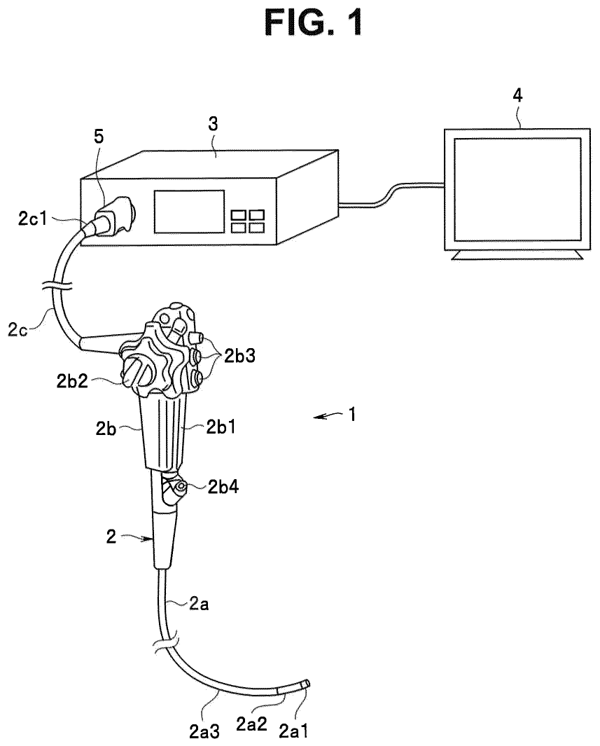

FIG. 1 to FIG. 8 show the first embodiment of the present disclosure. FIG. 1 is a view showing a configuration of an endoscope system 1 in the first embodiment.

The endoscope system 1 includes an endoscope 2, an endoscope processor 3, a monitor 4, and a relay adapter 5.

Note that, in the present embodiment, description will be made by taking the endoscope system 1, the endoscope 2, and the endoscope processor 3, as an example of a medical apparatus system, an example of a medical apparatus, and an example of a power supply apparatus configured to supply power to the medical apparatus, respectively. However, the medical apparatus system is not limited to the endoscope system 1. For example, the medical apparatus may be a treatment instrument such as a catheter. Therefore, the power supply apparatus is not limited to the endoscope processor 3.

The endoscope 2 is a single-use endoscope configured to be disposed of after having been used one time, for example. However, the endoscope 2 is not limited to the single-use endoscope.

The endoscope processor 3 is a processor for a reusable endoscope, which is used for a reusable endoscope, for example.

The single-use endoscope is sometimes different from a reusable endoscope in a standard of a connector (hardware standards such as connector shapes, and software standards such as signal standards, etc.). Hence, a configuration for enabling the single-use endoscope, which is different from the reusable endoscope in the standard of the connector, to be connected to the endoscope processor 3 is the relay adapter 5.

Note that the endoscope to be connected to the endoscope processor 3 using the relay adapter 5 is not limited to the single-use endoscope, but may be another reusable endoscope which is different in the standard of the connector from the reusable endoscope configured to be connectable to the endoscope processor 3.

The relay adapter 5 emulates the reusable endoscope connectable to the endoscope processor 3. The endoscope processor 3 recognizes the endoscope 2 that is a single-use endoscope, which has been connected to the endoscope processor 3 via the relay adapter 5, as the endoscope having the specifications connectable to the endoscope processor 3, and performs image processing or another processing. The relay adapter 5 may be a single-use adapter, but can be supposed to be a reusable adapter.

The endoscope 2 includes an insertion portion 2a, an operation portion 2b, and a universal cable 2c. The endoscope 2 is configured as an electronic endoscope, for example.

The insertion portion 2a is a part to be inserted into a subject. The subject is supposed to be a living thing such as a human being, an animal, or the like, for example. However, the subject may be non-living things such as machine, construction, or the like.

The insertion portion 2a includes, in the following order from the distal end side toward the proximal end side, a distal end portion 2a1, a bending portion 2a2, and a flexible tube portion 2a3.

In the distal end portion 2a1, an image pickup unit, a distal end portion of a light guide, and a distal end side opening of a treatment instrument channel, etc., are disposed. The image pickup unit includes an image pickup optical system and an image sensor. The image pickup optical system is configured of a plurality of lenses, an optical diaphragm, and the like, and forms an optical image of the subject on the image sensor. The image sensor photoelectrically converts (performs image pickup of) the optical image of the subject to generate an image pickup signal.

Examples of the image sensor include a CCD (charge coupled device) image sensor, a CMOS (complementary metal-oxide semiconductor) image sensor, and the like, but not limited to these.

The bending portion 2a2 is a part configured to be bendable in two directions, i.e., up and down directions, or in four directions, i.e., up, down, left, and right directions, for example.

The flexible tube portion 2a3 is a tubular member having flexibility. Note that description is made by taking a case where the endoscope 2 is a flexible endoscope having the flexible tube portion 2a3, as an example. However, the endoscope 2 may be a rigid endoscope in which the part corresponding to the flexible tube portion 2a3 is rigid.

The operation portion 2b is a part for a user to operate the endoscope 2. The operation portion 2b is disposed on the proximal end side of the insertion portion 2a. The operation portion 2b includes a grasping portion 2b1, a bending operation knob 2b2, a plurality of operation buttons 2b3, and a treatment instrument insertion port 2b4.

The grasping portion 2b1 is a part for the user to grasp the endoscope 2 with the palm.

The bending operation knob 2b2 is an operation device for operating bending of the bending portion 2a2. The bending operation knob 2b2 is operated by using the thumb of the hand grasping the grasping portion 2b1, for example. When the bending operation knob 2b2 is operated, the bending wire is pulled, to thereby cause the bending portion 2a2 to bend.

When the bending portion 2a2 is bent, the direction of the distal end portion 2a1 is changed. Then, an image pickup direction by the image pickup unit, and an irradiation direction of illumination light by the light guide are changed. In addition, the bending portion 2a2 is bent also for improving the insertion performance of the insertion portion 2a in the subject.

The plurality of operation buttons 2b3 include a gas/liquid feeding button, a suction button, and other buttons, for example. The gas/liquid feeding button is a button for the operation of feeding gas and liquid to an observation window provided on the distal end surface of the image pickup unit in the distal end portion 2al. The observation window is cleaned by liquid feeding, and the liquid remained after cleaning is blown off by gas feeding. The gas feeding and liquid feeding are performed through a gas/liquid feeding channel, not shown.

The suction button is a button for operation of sucking a liquid, a mucosa, and the like in the subject from the distal end portion 2a1. For example, the liquid, the mucosa, and the like are sucked through a suction channel provided in the endoscope 2.

The other buttons include a freeze button for temporarily stopping a monitor screen, a release button for image pickup of a still image, a switching button for switching light to special light, and the like, for example.

The treatment instrument insertion port 2b4 is an opening on the proximal end side of the treatment instrument channel. A treatment instrument such as a forceps is inserted into the treatment instrument channel from the treatment instrument insertion port 2b4. The distal end portion of the treatment instrument protrudes from the distal end side opening of the treatment instrument channel. Various kinds of treatment are performed on the subject with the distal end portion of the protruded treatment instrument.

The universal cable 2c is extended from the operation portion 2b. A first connector 2c1 is provided on the extension end of the universal cable 2c.

As described above, the first connector 2c1 of the single-use endoscope 2 has a standard different from that of the connector of the reusable endoscope.

The single-use endoscope 2 is connected to the endoscope processor 3 via the relay adapter 5.

The endoscope processor 3 supplies power to the endoscope 2 and transmits a control signal to the endoscope 2, via the relay adapter 5. In addition, the endoscope processor 3 receives an image pickup signal from the endoscope 2 via the relay adapter 5.

The endoscope processor 3 performs image processing on the image pickup signal received from the endoscope 2 to generate a video signal. The generated video signal is outputted to the monitor 4 and an endoscopic image is displayed on the monitor 4.

Note that description will be made below supposing that the endoscope processor 3 according to the present embodiment supplies the illumination light to the endoscope 2 via the relay adapter 5. However, a light source apparatus may be provided separately from the endoscope processor 3, or a configuration in which a light-emitting element such as an LED (light-emitting diode) may be provided at the distal end portion of the endoscope 2 to emit illumination light may be employed.

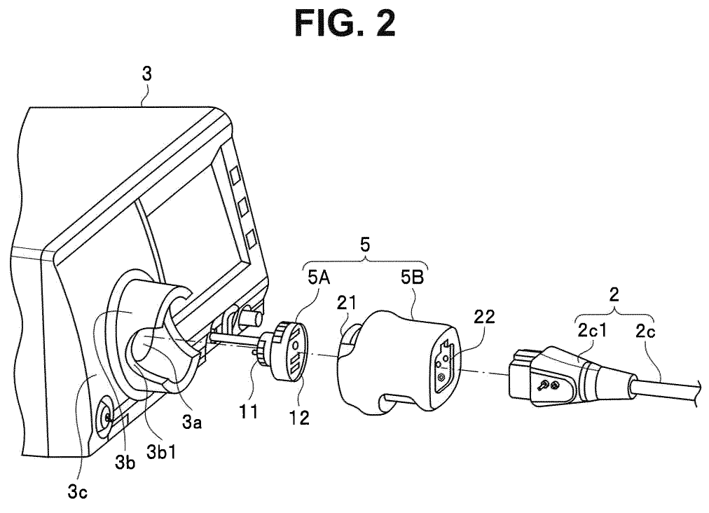

FIG. 2 is a partially exploded perspective view showing a configuration for connecting the endoscope 2 and the endoscope processor 3 by using a relay adapter 5 in the first embodiment.

The endoscope processor 3 includes a first receptacle 3a. The connector for reusable endoscope is connected to the first receptacle 3a. The first receptacle 3a is a connector receiver of a standard compliant with the standard of the connector for reusable endoscope.

The endoscope processor 3 includes, around the first receptacle 3a, a wall-like part 3b protruding like a wall from the surface (exterior surface) of an exterior member 3c of the endoscope processor 3. The wall-like part 3b includes a cutout part 3b1 which is cut out so that the protruding amount from the exterior surface of the endoscope processor 3 becomes small.

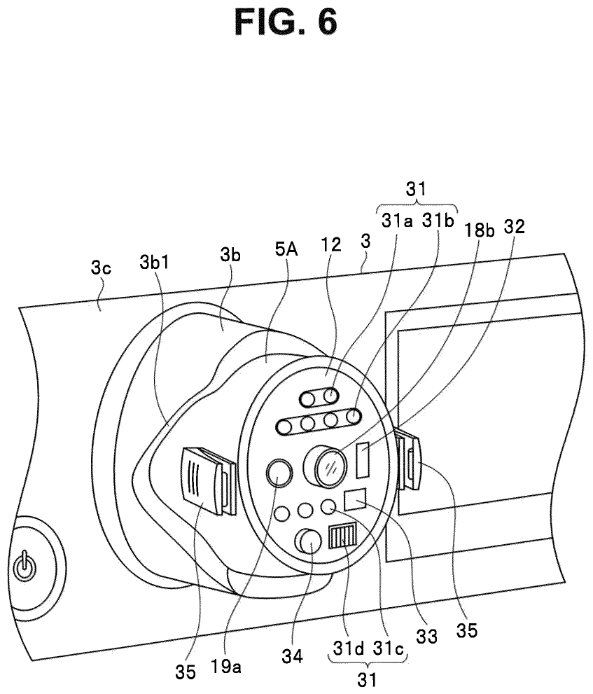

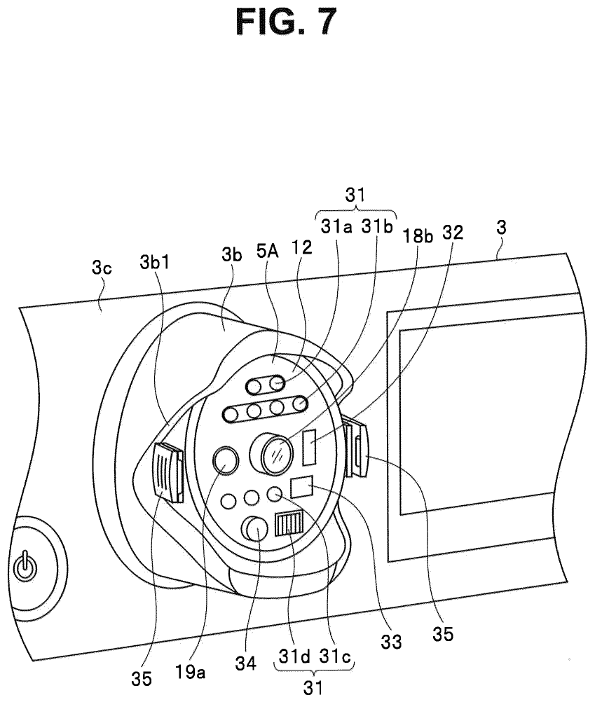

The wall-like part 3b surrounds at least a part of a side surface of the first adapter 5A connected to the first receptacle 3a (see FIG. 6 and FIG. 7, etc.).

The relay adapter 5 includes a first adapter 5A (primary adapter) and a second adapter 5B (secondary adapter). Hereinafter, a side of the endoscope processor 3 is referred to as one end side, and a side of the endoscope 2 is referred to as the other end side, as appropriate, when viewed from the relay adapter 5.

The first adapter 5A is connected, on the one end side (first end side) thereof, to the first receptacle 3a of the endoscope processor 3. The first adapter 5A includes, on the one end side thereof, a second connector 11 (first connector) configured to be detachably connected to the first receptacle 3a, and on the other end side (second end side) thereof, a first coupler 12.

The second connector 11 is a connector of a standard compliant with the standard of the connector for reusable endoscope to which the endoscope processor 3 is connectable.

The second adapter 5B is connected, on the one end side (first end side) thereof, to the other end side of the first adapter 5A, and on the other end side (second end side) thereof, to the first connector 2c1 of the endoscope 2. The second adapter 5B includes, on the one end side thereof, a second coupler 21 configured to be detachably connected to the first coupler 12, and on the other end side thereof, a second receptacle 22 configured to be detachably connected to the first connector 2c1.

The second receptacle 22 is a connector receiver of a standard compliant with the standard of the first connector 2c1 of the single-use endoscope 2.

Here, the single-use endoscope 2 may include a plurality of models of endoscopes 2 due to product development. In such a case, various types of first connectors 2c1 may be prepared in accordance with the plurality of models of the endoscopes 2.

In this case, a plurality of types of second adapters 5B corresponding to the respective plurality of types of first connectors 2c1 are prepared. Then, the second coupler 21 is configured to be common to the plurality of types of second adapters 5B. With such a configuration, one type of the first adapter 5A is selectively connectable to the second adapter 5B which is one of the plurality of types of the second adapter 5B. Thus, only one first adapter 5A is enough, which can reduce the introduction costs for the user.

The first coupler 12 and the second coupler 21 are configured such that plane portions thereof opposed to each other are combined at peripheral edges of the couplers, for example. The first coupler 12 and the second coupler 21 are connected to each other by wire via electric contacts, for example. When the plane portion of the first coupler 12 and the plane portion of the second coupler 21 are combined, the electric contacts and fluid connectors (flow channel ports) of the first and second couplers are combined. Note that the connection of the first and second couplers are not limited to the one using the electric contacts. The first coupler 12 and the second coupler 21 may be wirelessly connected.

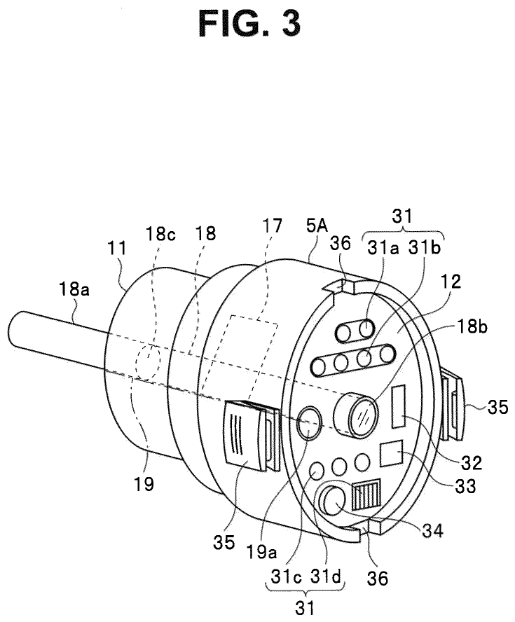

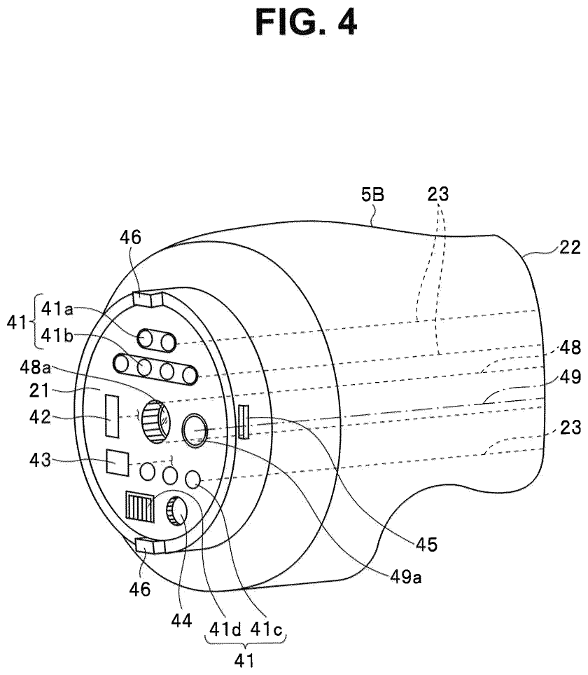

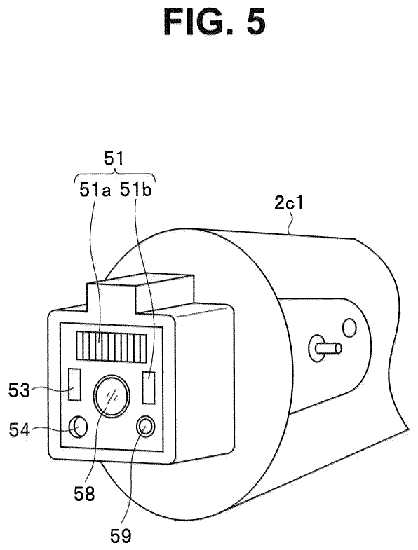

FIG. 3 is a perspective view showing the first coupler 12 side of the first adapter 5A in the first embodiment. FIG. 4 is a perspective view showing the second coupler 21 side of the second adapter 5B in the first embodiment. FIG. 5 is a perspective view showing the first connector 2c1 of the endoscope 2 in the first embodiment.

The first coupler 12 includes an electric contact 31 including electric contacts 31a, 31b, 31c, and 31d. Each of the electric contacts 31a, 31b, 31c, and 31d includes one or more electric contacts.

The second coupler 21 includes an electric contact 41 including electric contacts 41a, 41b, 41c, and 41d. The electric contact 41a corresponds to the electric contact 31a, and includes the same number of electric contacts as the electric contact 31a. The electric contact 41b corresponds to the electric contact 31b, and includes the same number of electric contacts as the electric contact 31b. The electric contact 41c corresponds to the electric contact 31c, and includes the same number of electric contacts as the electric contact 31c. The electric contact 41d corresponds to the electric contact 31d, and includes the same number of electric contacts as the electric contact 31d.

The second adapter 5B includes an electric wiring 23. The electric wiring 23 is connected to the electric contact 41.

The first connector 21cl includes an electric contact 51. The electric contact 51 includes an electric contact 51a and an electric contact 51b, for example. The first connector 2c1 is connected to the second receptacle 22, and thereby the electric contact 51 is connected to the electric wiring 23.

The first adapter 5A includes a first light guide body 18. One end side of the first light guide body 18 is protruded as a light guide portion 18a. The other end side of the first light guide body 18 is configured as an optical connector 18b. The first light guide body 18 transmits illumination light, which is supplied from the endoscope processor 3 via the second connector 11, to the first coupler 12.

The first adapter 5A includes an optical shutter 18c on an optical path of the illumination light transmitted by the first light guide body 18.

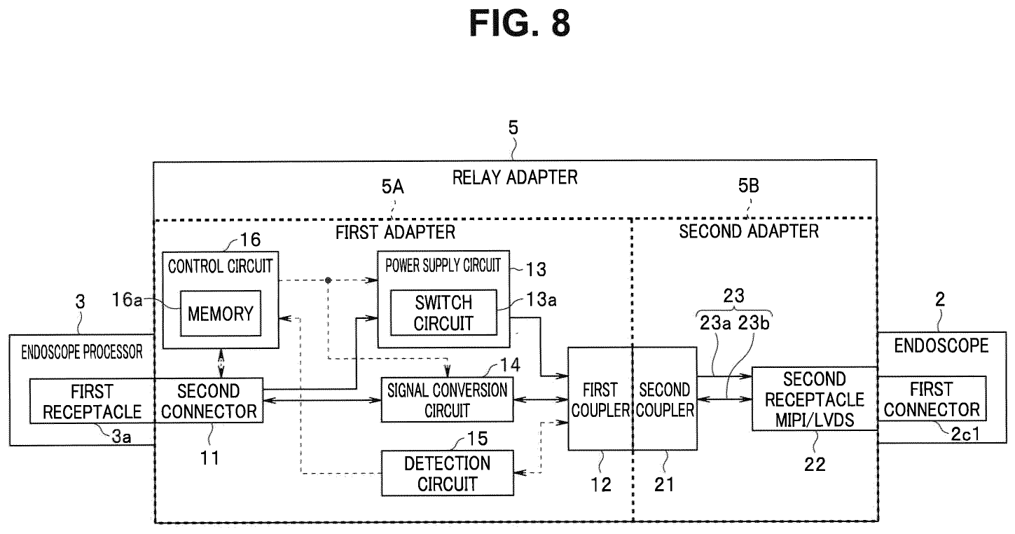

When a detection circuit 15 (see FIG. 8) to be described later detects that the first adapter 5A is not connected to the second adapter 5B, the optical shutter 18c blocks the illumination light transmitted from the second connector 11 to the first coupler 12, based on the control by a control circuit 16 (see FIG. 8).

When the detection circuit 15 detects that the first adapter 5A is connected to the second adapter 5B, the optical shutter 18c allows passage of the illumination light transmitted from the second connector 11 to the first coupler 12, based on the control by the control circuit 16.

The second adapter 5B includes a second light guide body 48. One end side of the second light guide body 48 is configured as an optical connector 48a. When the first coupler 12 and the second coupler 21 are connected, the optical connector 18b and the optical connector 48a are connected.

Although not shown, an optical connector is provided also on the other end side of the second light guide body 48. The second light guide body 48 transmits the illumination light, which is received from the first light guide body 18 via the second coupler 21, to the second receptacle 22.

The first connector 2c1 includes a light guide body 58. When the second receptacle 22 and the first connector 2c1 are connected, the optical connector on the other end side of the second light guide body 48 and the optical connector on the one end side of the light guide body 58 are optically connected. The illumination light transmitted via the first light guide body 18 and the second light guide body 48 is transmitted further to the distal end portion 2al of the endoscope 2 by the light guide body 58.

The first adapter 5A includes a first fluid conduit 19. The other end side of the first fluid conduit 19 is formed as a flow channel port 19a. Although not shown, a flow channel port is provided also on the one end side of the first fluid conduit 19. The first fluid conduit 19 is connected, at the flow channel port on the one end side, to a conduit provided in the first receptacle 3a of the endoscope processor 3.

The second adapter 5B includes a second fluid conduit 49. The one end side of the second fluid conduit 49 is formed as a flow channel port 49a. Although not shown, a flow channel port is provided also on the other end side of the second fluid conduit 49. When the first coupler 12 and the second coupler 21 are connected to each other, the flow channel port 19a and the flow channel port 49a are connected to each other.

The first connector 2c1 includes a fluid conduit 59. When the second receptacle 22 and the first connector 2c1 are connected to each other, the fluid conduit 59 and the second fluid conduit 49 are connected to each other.

The first fluid conduit 19 and the second fluid conduit 49 cause the fluid to flow between the fluid conduit 59 of the endoscope 2 and the endoscope processor 3.

However, each of the first receptacle 3a, the first adapter 5A, and the second adapter 5B does not have to include the conduit. The conduit of the endoscope 2 may be connected to the endoscope processor 3 (or another fluid supply system, etc.) through another channel different from the relay adapter 5.

The first adapter 5A includes a first detection contact 32. The second adapter 5B includes a first detection contact 42. The detection circuit 15 (see FIG. 8) to be described later detects that the second adapter 5B is connected to the first adapter 5A, when the first detection contact 32 and the first detection contact 42 are connected to each other, with the relay adapter 5 connected to the endoscope processor 3.

The first adapter 5A includes a second detection contact 33. The second adapter 5B includes a second detection contact 43. The first connector 2c1 includes a detection contact 53. The detection circuit 15 (see FIG. 8) to be described later detects that the endoscope 2 is connected to the relay adapter 5, when the second detection contact 33 and the second detection contact 43 are connected to each other, and the electric wiring 23 that is connected to the second detection contact 43 is connected to the detection contact 53, with the relay adapter 5 connected to the endoscope processor 3.

The first adapter 5A includes a pair of fixing levers 35, for example. The second adapter 5B includes a pair of protrusions 45 (claws) corresponding respectively to the pair of fixing levers 35. The pair of fixing levers 35 and the pair of protrusions 45 constitute a fixing tool (attachment mechanism) configured to detachably attach the first coupler 12 and the second coupler 21.

Note that the first adapter 5A is provided with the fixing levers 35 and the second adapter 5B is provided with the protrusions 45 in the present embodiment. However, the first adapter 5A may be provided with the protrusions and the second adapter 5B may be provided with the fixing levers.

Furthermore, in the present embodiment, the fixing levers 35 provided to the first adapter 5A and the protrusions 45 provided to the second adapter 5B are combined to configure the fixing tool. However, the configuration of the fixing tool is not limited to the above-described configuration. The fixing tool may be provided to at least one of the first adapter 5A and the second adapter 5B.

The first adapter 5A includes a pair of positioning shape portions 36, for example. The second adapter 5B includes a pair of positioning shape portions 46 corresponding respectively to the pair of positioning shape portions 36. The positioning shape portions 36 and 46 constitute a spline mechanism. The positioning shape portions 36 and 46 are configured to determine the positions of the first coupler 12 and the second coupler 21 in the rotational direction when the fixing levers 35 and the protrusions 45, which constitute the fixing tool, fix the first coupler 12 and the second coupler 21.

In the example shown in the drawings, each of the positioning shape portions 36 has a recessed shape portion and each of the positioning shape portions 46 has a projected shape portion, but the recessed shape and the projected shape in the positioning shape portions 36 and 46 may be reversed. Note that the positioning shape portions may be provided to at least one of the first adapter 5A and the second adapter 5B.

The first coupler 12 includes a projected shape portion 34. The second coupler 21 includes a recessed shape portion 44. The projected shape portion 34 and the recessed shape portion 44 are fitted to each other when the fixing levers 35 and the protrusions 45, which constitute the fixing tool, fix the first coupler 12 and the second coupler 21, to prevent a backlash between the first coupler 12 and the second coupler 21.

The first connector 2c1 includes a recessed shape portion 54. The recessed shape portion 54 is fitted with a projected shape portion, not shown, formed in the second receptacle 22, to thereby prevent a backlash between the second receptacle 22 and the first connector 2cl. Note that, in the example shown in the drawings, the first connector 2c1 includes the recessed shape portion 54 and the second receptacle 22 includes the projected shape portion, but the recessed shape and the projected shape in the first connector 2c1 and the second receptacle 22 may be reversed.

FIG. 6 is a perspective view showing a first example in which only the first adapter 5A of the relay adapter 5 is connected to a first receptacle 3a in the first embodiment. FIG. 7 is a perspective view showing a second example in which only the first adapter 5A of the relay adapter 5 is connected to the first receptacle 3a in the first embodiment.

The first adapter 5A in the first example shown in FIG. 3 is configured such that the thickness thereof in the insertion direction (direction connecting the one end side and the other end side) is larger than the thickness of the first adapter 5A in the second example shown in FIG. 2 and FIG. 7.

The first example shown in FIG. 6 shows a state where the first adapter 5A shown in FIG. 3 is connected to the first receptacle 3a. In the first example shown in FIG. 6, the other end side of the first adapter 5A including the first coupler 12 protrudes further than a protrusion end of the wall-like part 3b.

In the first example shown in FIG. 6, the first adapter 5A can be easily detached from the first receptacle 3a, with only the first adapter 5A connected to the first receptacle 3a. In addition, in the first example, the second adapter 5B can be easily connected to the first adapter 5A, with only the first adapter 5A connected to the first receptacle 3a. Furthermore, in the first example, only the second adapter 5B in the relay adapter 5 can be easily detached or replaced (detached and another second adapter 5B is connected), with the relay adapter 5 connected to the first receptacle 3a.

The second example shown in FIG. 7 shows a state where the first adapter 5A shown in FIG. 2 is connected to the first receptacle 3a. In the second example shown in FIG. 7, the other end side of the first adapter 5A including the first coupler 12 is recessed inwardly from the protrusion end of the wall-like part 3b.

In other words, in the second example shown in FIG. 7, the maximum protruding amount of the wall-like part 3b from the surface (exterior surface) of the exterior member 3c is larger than the maximum protruding amount of the first adapter 5A, which is connected by itself to the first receptacle 3a, protruding from the surface (exterior surface) of the exterior member 3c.

In the second example shown in FIG. 7, the first adapter 5A does not protrude from the protrusion end of the wall-like part 3b, which reduces the possibility of restricting the space required for the procedure in the endoscopy room in preparation for the procedure, or the like, compared with the first example.

As shown in FIG. 3, the first adapter 5A includes an electric circuit substrate 17. FIG. 8 is a block diagram showing an electrical configuration of the relay adapter 5 in the first embodiment.

The first adapter 5A includes a power supply circuit 13, a signal conversion circuit 14, a detection circuit 15, and a control circuit 16, in addition to the second connector 11 and the first coupler 12.

The second adapter 5B includes an electric wiring 23 including electric wirings 23a, 23b, in addition to the second coupler 21 and the second receptacle 22.

The respective circuits of the first adapter 5A are operable when the second connector 11 is connected to the first receptacle 3a. The respective circuits of the second adapter 5B are operable when the second connector 11 is connected to the first receptacle 3a and the second coupler 21 is connected to the first coupler 12. The respective circuits of the endoscope 2 are operable when the second connector 11 is connected to the first receptacle 3a and the second coupler 21 is connected to the first coupler 12, and the first connector 2c1 is connected to the second receptacle 22.

On an electric circuit substrate 17 shown in FIG. 3, the power supply circuit 13, the signal conversion circuit 14, the detection circuit 15, and the control circuit 16 are mounted, for example.

The control circuit 16 includes a memory 16a. The memory 16a includes a rewritable non-volatile memory, for example. Note that the memory 16a is provided in the control circuit 16 in the example shown in FIG. 8. However, the memory 16a may be provided separately from the control circuit 16.

The control circuit 16 is configured to execute functions by a processor, such as an ASIC (Application Specific Integrated Circuit) including a CPU (Central Processing Unit), or FPGA (Field Programmable Gate Array, for example, reading and executing a processing program stored in a storage apparatus (or a recording medium) such as the memory 16a. However, the control circuit 16 may be configured such that each of the functions thereof is executed by a dedicated electronic circuit.

The memory 16a stores information for the single-use endoscope 2, which is connected to the relay adapter 5, to emulate the reusable endoscope connectable to the endoscope processor 3.

The single-use endoscope 2 includes a plurality of models in some cases, and also the reusable endoscope connectable to the endoscope processor 3 includes a plurality of models in some cases. Therefore, the memory 16a stores information for allowing one or more models of single-use endoscopes 2 to emulate one or more models of reusable endoscopes.

Specifically, the memory 16a stores information such as ID information (a model number indicating a model, a serial number discriminating an individual, etc.), a standard, and the like of at least one model of reusable endoscope connectable to the endoscope processor 3. In addition, the memory 16a stores information such as ID information, standards, and the like of one or more models of single-use endoscope.

Furthermore, the memory 16a stores, for example as a table, setting information according to a combination of the model (ID information) of the single-use endoscope and the model (ID information) of the reusable endoscope. Note that if the relay adapter 5 is configured to emulate only one model of reusable endoscope, the memory 16a may store a table corresponding to the model of the single-use endoscope.

The setting information stored in the table by the memory 16a includes information for setting the power supply circuit 13 and information setting the signal conversion circuit 14.

The control circuit 16 functions also as a communication unit configured to communicate with the endoscope 2 and the endoscope processor 3. The control circuit 16, which functions also as the communication unit, is configured to absorb differences in communication between the endoscope 2 and the endoscope processor 3, to thereby enable commonality of the communication.

The single-use endoscope 2, in general, includes a memory for storing ID information (furthermore, setting values, information on an image sensor, etc., as needed), a circuit configured to transmit the ID information, etc.

The control circuit 16 functions as a communication unit, and acquires the ID information and the like from the single-use endoscope 2. The control circuit 16 refers to a table in the memory 16a according to the ID information of the endoscope 2, to determine one model of reusable endoscope to be emulated.

The control circuit 16 transmits the ID information and the like of the reusable endoscope which is determined to be emulated to the endoscope processor 3. When the endoscope 2 is connected to the endoscope processor 3 via the relay adapter 5, the endoscope processor 3 determines that the reusable endoscope to be emulated has been connected. Then, the endoscope processor 3 supplies power and a control signal conforming to the reusable endoscope to be emulated to the relay adapter 5.

In addition, the control circuit 16 acquires, from the table in the memory 16a, the setting information based on which the endoscope 2 emulates the determined one model of reusable endoscope.

The control circuit 16 functions as a setting unit, and sets the power supply circuit 13 and the signal conversion circuit 14 based on the acquired setting information.

Assume that lines of voltage of power adapted to the reusable endoscope to be emulated are one with 1.2V and two with 1.8V, for example. On the other hand, assume that lines of voltage of power adapted to the single-use endoscope are two with 1.0V and one with 1.2V, for example.

Then, the control circuit 16 sets the power supply circuit 13 so as to convert an input of voltage of which lines are one with 1.2V and two with 1.8V into an output of voltage of which lines are two with 1.0V and one with 1.2V.

The endoscope processor 3 supplies power adapted to the emulated reusable endoscope. The power supply circuit 13 converts, according to the setting, the power transmitted from the endoscope processor 3 (voltage, current, the number of lines) into power adapted to the single-use endoscope 2. The converted power is transmitted to the first connector 2c1 of the endoscope 2, via the first coupler 12, the second coupler 21, the electric wiring 23a, and the second receptacle 22.

The control circuit 16 further sets the signal conversion circuit 14 based on the acquired setting information. Then, the signal conversion circuit 14 converts a signal transmitted from the endoscope processor 3 into a signal adapted to the endoscope 2, to transmit the converted signal to the endoscope 2. Furthermore, the signal conversion circuit 14 converts the signal transmitted from the endoscope 2 into a signal adapted to the endoscope processor 3, to transmit the converted signal to the endoscope processor 3.

Specifically, the signal conversion circuit 14 converts the control signals (control signals including a clock signal, a vertical synchronizing signal, a horizontal synchronizing signal, etc., which are transmitted to the reusable endoscope) transmitted from the endoscope processor 3 into control signals adapted to the endoscope 2, and transmits the converted control signal to the endoscope 2. Then, the image sensor included in the endoscope 2 generates an image pickup signal, to transmit the generated image pickup signal to the signal conversion circuit 14.

Furthermore, the control circuit 16 communicates with the endoscope processor 3, to acquire information on white balance setting from the endoscope processor 3. The control circuit 16 controls the signal conversion circuit 14 based on the information on the white balance setting, to perform white balance adjustment. Information required for the signal conversion circuit 14 to perform the white balance adjustment is stored in the memory 16a. Such a configuration eliminates a need for providing a circuit that performs the white balance adjustment in the endoscope 2, which enables the price reduction.

In addition, a storage capacity of the memory in the endoscope 2 can be reduced by separating the information to be stored in the memory of the endoscope 2 from the information to be stored in the memory 16a of the first adapter 5A. This enables further price reduction of the endoscope 2.

The signal conversion circuit 14 performs signal conversion such that a format (number of pixels, frame rate, etc.) of the image pickup signal generated by the image sensor provided in the single-use endoscope 2 is converted into a format of the image pickup signal generated by the image sensor provided in the reusable endoscope to be emulated. The signal conversion circuit 14 transmits the converted image pickup signal to the endoscope processor 3.

The control signal and the image pickup signal are transmitted by the electric wiring 23b in the second adapter 5B.

The detection circuit 15 detects, as described above, whether the second adapter 5B is connected to the first adapter 5A, depending on whether the first detection contact 32 and the first detection contact 42 are connected to each other.

When the first detection contact 32 and the first detection contact 42 are connected to each other, the second detection contact 33 and the second detection contact 43 are connected to each other. In this case, the detection circuit 15 detects whether the endoscope 2 is connected to the relay adapter 5, depending on whether the electric wiring 23 connected to the second detection contact 43 is connected to the detection contact 53.

When the first adapter 5A is not connected to the endoscope processor 3, the detection circuit 15 does not operate and does not transmit a detection result to the control circuit 16. When the first adapter 5A is connected to the endoscope processor 3, the detection circuit 15 operates and transmits a detection result to the control circuit 16.

The detection result transmitted by the detection circuit 15 is any one of the following: (1) the second adapter 5B is not connected to the first adapter 5A; (2) the second adapter 5B is connected to the first adapter 5A, and the endoscope 2 is not connected to the second adapter 5B; and (3) the second adapter 5B is connected to the first adapter 5A and the endoscope 2 is connected to the second adapter 5B.

The power supply circuit 13 includes a switch circuit 13a that is controlled by the control circuit 16.

When the detection result transmitted from the detection circuit 15 is (1) or (2), the control circuit 16 causes the switch circuit 13a to stop the supply of power from the power supply circuit 13 to the endoscope 2. This prevents current from flowing to the electric contact 31 of the first coupler 12 and the electric wiring 23 of the second receptacle 22.

In addition, when the detection result transmitted from the detection circuit 15 is (3), the control circuit 16 causes the switch circuit 13a to allow the supply of power from the power supply circuit 13 to the endoscope 2.

Thus, the switch circuit 13a is provided, to thereby increase the safety of the relay adapter 5 and prevent malfunction thereof.

Note that FIG. 8 shows the example in which the switch circuit 13a is provided in the power supply circuit 13, but the switch circuit 13a may be provided outside the power supply circuit 13 as long as the switch circuit 13a is located on a power supply route in the first adapter 5A.

For example, there is a case where a new model of the single-use endoscope 2 is manufactured to be sold, by updating the image sensor mounted to the single-use endoscope 2. The relay adapter 5 can be configured to be capable of supplying power adapted to various models within an envisioned range of the power for the new model of the single-use endoscope 2.

Meanwhile, when a new model of the single-use endoscope 2 is developed, it can be that an image sensor and the like to which the relay adapter 5 can supply power are employed in the endoscope 2.

Then, the table stored in the rewritable memory 16a is updated to a table corresponding to the ID information of the new model of the endoscope 2.

With such a configuration, the relay adapter 5 can supply power to the new model of the endoscope 2 based on the ID information, by simply upgrading the software without changing the hardware. Therefore, there is no need for preparing a relay adapter 5 for each model of the endoscope 2, which reduces the introduction costs for the user.

As described above, the first adapter 5A includes a circuit of a first circuit scale (size of a first circuit) including the power supply circuit 13, the signal conversion circuit 14, the detection circuit 15, and the control circuit 16. The second adapter 5B includes a circuit of a second circuit scale (size of a second circuit) including the electric wiring 23. The first circuit scale of the first adapter 5A is larger than the second circuit scale of the second adapter 5B. In other words, the circuit of the first adapter 5A is more complicated than the circuit of the second adapter 5B. For example, the circuit of the first adapter 5A includes more important circuits for determination or signal processing than the circuit of the second adapter 5B. For other example, a number of individual electrical elements included in the circuit of the first adapter 5A is larger than a number of individual electrical elements included in the circuit of the second adapter 5B. The individual electrical elements include wiring, resistors, memory, controllers and others.

The first adapter 5A includes more important electric circuits and is more expensive to manufacture, compared with the second adapter 5B. Therefore, the first adapter 5A may be configured such that the product life thereof is longer than that of the second adapter 5B.

For example, the first adapter 5A may be disposed of after the first maximum number of usages, and the second adapter 5B may be disposed of after the second maximum number of usages. At this time, the first maximum number of usages may be larger than the second maximum number of usages. Note that, since the relay adapter 5 including the second adapter 5B is supposed to be a re-use type (to be used a plurality of times) as described above, the first maximum number of usage and the second maximum number of usages are each plural numbers. The first maximum number of usages may be more than a thousand times. The second maximum number of usages may be less than a hundred times. The second maximum number of usages may be less than a few hundred times.

In order to achieve such a configuration, among the components constituting the first adapter 5A, components with the shortest product life have a product life that withstands the first maximum number of usages. In addition, among the components constituting the second adapter 5B, components with the shortest product life have a product life that withstands the second maximum number of usages.

Examples of the components with the shortest product life include the electric contacts 31, 41, and the fixing levers 35 and the protrusions 45 that constitute the fixing tool, and the like. Therefore, if the product life of the first adapter 5A is made to be longer than the product life of the second adapter 5B, it can be that the product life of the electric contact 31 may be made to be longer than that of the electric contact 41, and the product life of the fixing levers 35 may be made to be longer than that of the protrusions 45. In order to make the product life longer, known methods may appropriately be used, such as using a high wear-resistant material and/or coating for relevant components.

Note that, among the components constituting the endoscope processor 3, the component with the shortest product life may have a product life that withstands the number of usages which is larger than the first maximum number.

As described above, the first connector 2c1 of the endoscope 2 includes a plurality of types, in some cases. However, in order to achieve price reduction of the endoscope 2, a general-purpose connector and a general-purpose signal standard may be used for the first connector 2cl, for example.

Specifically, the first connector 2c1 is configured as a USB (universal serial bus) connector, for example, and as the signal standard, a camera transmission standard (MIPI: mobile industry processor interface) is used, for example. Using the MIPI makes it easy to update image sensors, for example.

The second receptacle 22 corresponds to the first connector 2c1. The second receptacle 22 is configured as an USB receptacle, for example, and uses the MIPI as the signal standard.

Note that the signal standard for each of the first connector 2c1 and the second receptacle 22 is not limited to the MIPI, but a low voltage differential signal (LVDS: low voltage differential signaling) or the like may be used.

The general-purpose connector and the general-purpose signal standard are continuously used also for a new model of endoscope 2 in which the image sensor is updated, to thereby enable not only the first adapter 5A but also the second adapter 5B to be used, without changing the hardware.

Then, when a single-use endoscope 2 of a certain model is connected to the endoscope processor 3 and thereafter a single-use endoscope 2 of a different model is connected to the endoscope processor 3, there is no need for replacing the relay adapter 5, which saves efforts of the user.

According to the first embodiment, the relay adapter 5 is configured to include the first adapter 5A and the second adapter 5B. Therefore, in the application using a plurality of endoscopes for a plurality of cases in one day, only the first adapter 5A is left inserted in the first receptacle 3a of the endoscope processor 3, to thereby be capable of suppressing the protrusion of the adapter from the endoscope processor 3.

Such a configuration can prevent the adapter from restricting the space required for the procedure, in the case where the endoscopy room is narrow.

In addition, the first adapter 5A is left inserted in the first receptacle 3a of the endoscope processor 3, to thereby decrease the number of times of attaching and detaching the first adapter 5A to and from the first receptacle 3a even in the case where the endoscope 2 is used for a large number of cases. This can suppress the degradation of the first receptacle 3a.

The control circuit 16 that functions as the setting unit is configured to set the power supply circuit 13 and the signal conversion circuit 14 according to the model and the like of the endoscope 2, to thereby be capable of ensuring compatibility of the endoscope 2 with respect to the endoscope processor 3.

The relay adapter 5 includes the power supply circuit 13, which eliminates a need for the single-use endoscope 2 to include a circuit for converting the power supplied from the endoscope processor 3. This enables simplification of the configuration and further price reduction of the endoscope 2.

The relay adapter 5 of the present embodiment is capable of lowering the barrier of development of a new model of single-use endoscope 2, to facilitate the introduction of single-use endoscopes 2 to the market.

In addition, in facilities already equipped with the endoscope processor 3 for reusable endoscope, in a case where there is a desire for using the single-use endoscope 2, it is not necessary to purchase an endoscope processor dedicated to single-use endoscope 2, which can reduce the initial investment of the user.

Furthermore, the common relay adapter 5 can be used for a plurality of models of endoscopes 2, without a need for preparing a relay adapter for each of the models of endoscope 2, to thereby be capable of achieving a simpler endoscope system 1.

Note that the present disclosure is not limited as-is to the above described embodiment. It is possible to embody the present disclosure by modifying the constituent elements in a range without departing from the gist of the invention at the practical stage. In addition, various aspects of the disclosure can be achieved by appropriately combining the plurality of constituent elements disclosed in the above-described embodiment. Some of the constituent elements may be deleted from all the constituent elements shown in the embodiment, for example.

Furthermore, constituent elements over different embodiments may be combined as appropriate. It goes without saying that various modifications and applications can be implemented within a range without departing from the gist of the disclosure.

What is claimed is:

1. A relay adapter, comprising:
a first adapter including:
a first end side configured to connect to a first receptacle of a power supply apparatus,
a second end side,
a power supply circuit configured to supply power transmitted from the power supply apparatus to the second end side, and
a detection circuit; and
a second adapter including:
a first end side configured to connect to the second end side of the first adapter,
a second end side configured to connect to a connector of a medical apparatus, and
an electric circuit configured to transmit power supplied to the first end side of the second adapter from the first adapter to the connector of the medical apparatus,
wherein the detection circuit is configured to detect whether the second end side of the first adapter is connected to the first end side of the second adapter.

2. The relay adapter according to claim 1, wherein the first adapter includes a first connector on the first end side, and the first end side of the first adapter is configured to connect to the first receptacle of the power supply apparatus by the first connector connecting to the first receptacle of the power supply apparatus,
wherein the first adapter includes a first coupler on the second end side,
wherein the second adapter includes a second coupler on the first end side, and the first end side of the second adapter is connected to the second end side of the first adapter by the second coupler connecting to the first coupler, and
wherein the second adapter includes a second receptacle on the second end side, and the second end side of the second adapter is configured to connect to the connector of the medical apparatus by the second receptacle connecting to the connector of the medical apparatus.

3. The relay adapter according to claim 2, wherein the second coupler is connected to the first coupler by a wired connection including an electrical surface contact.

4. The relay adapter according to claim 2, wherein the second coupler is connected to the first coupler by a wirelessly connection.

5. The relay adapter according to claim 2, wherein the first adapter includes a first light guide body configured to receive an illumination light supplied from the power supply apparatus via the first connector and to transmit the illumination light from the first connector to the first coupler, and
wherein the second adapter includes a second light guide body configured to receive the illumination light from the first light guide body via the second coupler and to transmit the illumination light from the second coupler to the second receptacle.

6. The relay adapter according to claim 5, wherein the first adapter further includes an optical shutter configured to block the illumination light transmitted from the first connector to the first coupler when the detection circuit detects that the second end side of the first adapter is not connected to the first end side of the second adapter.

7. The relay adapter according to claim 2, wherein at least one of the first adapter and the second adapter includes an attachment mechanism configured to detachably attach the first coupler and the second coupler.

8. The relay adapter according to claim 7, wherein at least one of the first adapter and the second adapter includes a spline mechanism configured to rotationally position the first coupler relative to the second coupler when the first coupler and the second coupler are attached to each other by the attachment mechanism.

9. The relay adapter according to claim 1, wherein the first adapter further includes a switch circuit configured to stop supplying power from the power supply circuit to the medical apparatus when the detection circuit detects that the second end side of the first adapter is not connected to the first end side of the second adapter.

10. The relay adapter according to claim 1, wherein the first adapter includes a first circuit that includes the power supply circuit and the detection circuit,
wherein the second adapter includes a second circuit that includes electric wiring, and
wherein a size of the first circuit is larger than a size of the second circuit.

11. The relay adapter according to claim 1, wherein the connector of the medical apparatus includes a plurality of connector types, and the second end side of the second adapter includes a plurality of second adapter types,
wherein a respective one of the plurality of second adapter types connects to a respective one of the plurality of connector types, and
wherein the second end side of the first adapter is connectable to one of the plurality of second adapter types.

12. The relay adapter according to claim 1, wherein the medical apparatus is a single-use endoscope.

13. The relay adapter according to claim 12, wherein the first adapter is disposed of after a first maximum number of usages,
wherein the second adapter is disposed of after a second maximum number of usages, where the second maximum number of usages is more than two, and
wherein the first maximum number of usages is larger than the second maximum number of usages.

14. The relay adapter according to claim 13, wherein, amongst the plurality of components constituting the first adapter, a first adapter component with a shortest product life has a product life that is larger than the first maximum number of usages, and
wherein, amongst the plurality of components constituting the second adapter, a second adapter component with a shortest product life has a product life that is larger than the second maximum number of usages.

15. A medical apparatus system, comprising:
a medical apparatus including a connector;
a power supply apparatus including a first receptacle, the power supply apparatus configured to transmit power to be supplied to the medical apparatus; and
a relay adapter comprising:
a first adapter including:
a first end side configured to connect to the first receptacle,
a second end side,
a power supply circuit configured to supply power transmitted from the power supply apparatus to the second end side, and
a detection circuit, and
a second adapter including:
a first end side configured to connect to the second end side of the first adapter,
a second end side configured to connect to a connector of a medical apparatus, and
an electric circuit configured to transmit power supplied to the first end side of the second adapter from the first adapter to the connector of the medical apparatus,
wherein the detection circuit is configured to detect whether the second end side of the first adapter is connected to the first end side of the second adapter.

16. The medical apparatus system according to claim 15, wherein the power supply apparatus includes a protrusion located around at least a portion of a periphery of the first receptacle and extending outward from an exterior surface of a body of the power supply apparatus,
wherein a body of the protrusion includes a cutout region, a protruding length of the cutout region from the exterior surface being a first length, a protruding length of the protrusion in a region other than the cutout region from the exterior surface being a second length, and the first length being less than the second length, and
wherein, when the first end side of the first adapter is connected to the first receptacle of the power supply apparatus, the protrusion surrounds at least a portion of a side surface of the first end side of the first adapter.

17. The medical apparatus system according to claim 16, wherein, when the first end side of the first adapter is connected to the first receptacle of the power supply apparatus:
a maximum protruding amount of the first adapter is a third length, and
a maximum protruding length of the protrusion from the exterior surface is larger than the third length.

18. The medical apparatus system according to claim 15, wherein the medical apparatus is a single-use endoscope.

19. The medical apparatus system according to claim 18, wherein the first adapter is disposed of after a first maximum number of usages,
wherein the second adapter is disposed of after a second maximum number of usages, where the second maximum number of usages is more than two, and
wherein the first maximum number of usages is larger than the second maximum number of usages.

20. The medical apparatus system according to claim 19, wherein, amongst the plurality of components constituting the first adapter, a first adapter component with a shortest product life has a product life that is larger than the first maximum number of usages,
wherein, amongst the plurality of components constituting the second adapter, a second adapter component with a shortest product life has a product life that is larger than the second maximum number of usages, and
wherein, amongst the plurality of components constituting the power supply apparatus, a power supply apparatus component with a shortest product life has a product life that is larger than the first maximum number of usages.
